(19) United States
(12) Patent Application Publication
(10) Pub. No.: US 2007/0149815 A1
(43) Pub. Date: Jun. 28, 2007

(54) Organic light-emitting material and method for producing an organic material

(76) Inventors: Ichinori Takada, Tokyo (JP); Naoyuki Ueda, Tokyo (JP)

Correspondence Address: BELL, BOYD & LLOYD, LLP, P. O. BOX 1135, CHICAGO, IL, 60690, US

(21) Appl. No.: 10/595,710

(22) PCT Filed: Nov. 5, 2004
(86) PCT No.: PCT/JP04/16803

§ 371 Date: May 25, 2006

(30) Foreign Application Priority Data
Nov. 7, 2003 (JP) P2003-377904
Sep. 2, 2004 (JP) P2004-255344
Oct. 29, 2004 (JP) P2004-315486

(51) Int. Cl. C07C 211/60; C07C 211/61; C09K 11/06; H01L 51/54

(52) U.S. Cl. 564/426; 564/434; 564/404; 564/395; 257/E51.051; 257/E51.049; 313/504; 428/917

(57) ABSTRACT

An organic light-emitting material characterized in that it is used in a light emitting layer in a green light emitting element and represented by the following general formula (1): 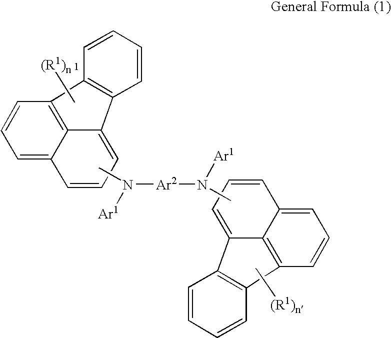
wherein: n¹ is an integer of 0 to 3; R¹ is an alkyl group having 10 carbon atoms or less; Ar¹ is a monovalent group which is derived from monocyclic or fused-ring aromatic hydrocarbon having 20 carbon atoms or less, and which optionally has a substituent having 10 carbon atoms or less; and Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less. There can be provided an organic light-emitting material which has satisfactorily excellent light emission efficiency and high color purity as well as higher reliability and which is advantageously used to constitute a green light emitting layer, and a method for producing the same.

### TECHNICAL FIELD

The present invention relates to an organic light-emitting material and a method for producing the same. More particularly, the present invention is concerned with an organic light-emitting material which is added to a light emitting layer in a light emitting element to cause the layer to emit green light, and a method for producing an organic material.

### BACKGROUND ART

An organic EL display is a display device comprising organic EL elements arranged as light emitting elements, and can provide clear images and can be reduced in thickness and hence has attracted attention as a candidate for next-generation flat panel display. However, for bringing the organic EL display into practical use, it is essential to improve the organic EL element in light emission efficiency and emission lifetime. Under the circumstances, for the purpose of improving the organic EL element in light emission efficiency and light emission luminance, a construction comprising a layer containing a benzofluoranthene derivative sandwiched between a pair of electrodes has been proposed (see Japanese Patent Application Publication Nos. 2002-69044, 2002-43058, and HEI10-189247).

In the organic EL display using the organic EL element, for realizing full color display, the use of light emitting materials of three primary colors (red, green, and blue) having high light emission efficiency and high color purity as well as high reliability is indispensable. Of the light emitting materials of three colors, the green light emitting material has been studied the most thoroughly, and materials having a basic laser dye skeleton, such as coumarin and quinacridone, are being developed (see U.S. Pat. Nos. 4,736,032 and 5,593,788).

### DISCLOSURE OF THE INVENTION

The most important task of putting the organic EL display on the market is to obtain an element having high reliability. The organic light-emitting material in the element is, however, under severe conditions such that a cycle of excitation and deactivation is repeated, and therefore part of the organic materials constituting the element inevitably suffer a chemical reaction, and thus an organic EL display having satisfactory light emission efficiency and satisfactory reliability has not yet been obtained.

Accordingly, a task of the present invention is to provide an organic light-emitting material emitting green light and having satisfactorily excellent light emission efficiency and high color purity as well as higher reliability, and a method for producing the same.

The first organic light-emitting material of the present invention for attaining the above task is characterized in that it is used in a light emitting layer in a green light emitting element (e.g., organic EL element) and represented by the following general formula (1):

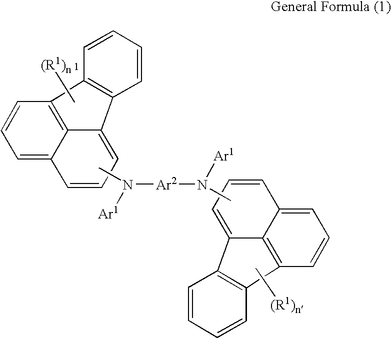

In the general formula (1) above, n¹ is an integer of 0 to 3, and R¹ is an alkyl group having 10 carbon atoms or less. When n¹ is 2 or 3 and each fluoranthene is substituted with two or three R¹'s at a plurality of positions (carbon atoms numbered), each of the R¹'s may be independently an alkyl group having 10 carbon atoms or less. Ar¹ is a monovalent group derived from monocyclic or fused-ring aromatic hydrocarbon having 20 carbon atoms or less, and may have a substituent having 10 carbon atoms or less. Ar² is a divalent group derived from a ring assembly having 30 carbon atoms or less and being comprised of monocyclic or fused-ring aromatic hydrocarbon having 1 to 3 rings. The divalent group may have a substituent having 4 carbon atoms or less.

The second organic light-emitting material of the present invention is an organic light-emitting material represented by the following general formula (2):

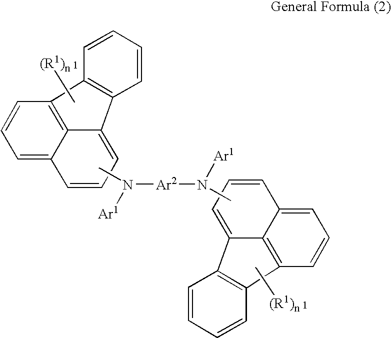

The general formula (2) is similar to the general formula (1) above, and n¹, R¹, Ar¹, and Ar² are similar to those defined in the general formula (1) above. However, in the second organic light-emitting material, in the general formula (2), the case where the monovalent group constituting Ar¹ is an unsubstituted phenyl group, the divalent group constituting Ar² is a divalent group derived from unsubstituted biphenyl, and each of two fluoranthenes is bonded to nitrogen at the carbon numbered 3 is excluded.

The second organic light-emitting material is a light emitting material used in a light emitting layer in a green light emitting element (e.g., organic EL element).

Each of the first organic light-emitting material and the second organic light-emitting material of the present invention having the above-described construction has a very strong molecular skeleton comprised of 3 constituent elements. In other words, Alq3 conventionally widely used as an organic light-emitting material emitting green light is comprised of 5 constituent elements (carbon, hydrogen, oxygen, nitrogen, and aluminum). In addition, many conventional organic light-emitting materials emitting green light including coumarin and quinacridone are comprised of 4 constituent elements or more. The number of constituent elements of the organic light-emitting material of the present invention is 3, which is small, as compared to that of the conventional organic light-emitting material emitting green light, and thus a stronger molecular skeleton is achieved. Therefore, the organic light-emitting material of the present invention has such a high resistance as an organic light-emitting material emitting green light that it is prevented from deteriorating. In addition, when the organic light-emitting material is used in a green light emitting layer, a light emitting element (e.g., organic EL element) having high chromaticity and high luminance is formed.

Furthermore, the present invention is directed to a method for producing an organic material represented by the general formula (3) below including both the above-described first organic light-emitting material and second organic light-emitting material.

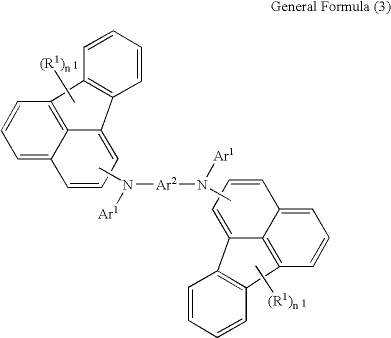

The general formula (3) is similar to the general formula (1) and general formula (2) above, and n¹, R¹, Ar¹, and Ar² are similar to those defined in the general formula (1) and general formula (2) above, and the case where the monovalent group constituting Ar¹ is an unsubstituted phenyl group and the divalent group constituting Ar² is a divalent group derived from unsubstituted biphenyl is included.

In the method of the present invention, the first method is a method for producing the organic material, comprising reacting a compound represented by the general formula (4)-1 below with a compound represented by the general formula (4)-2 below using a metal catalyst. As the metal catalyst, a palladium catalyst or a copper catalyst may be used.

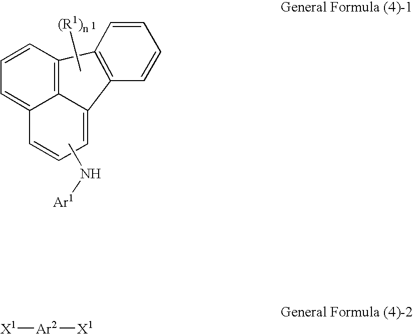

n¹, R¹, Ar¹, and Ar² in the general formula (4)-1 and general formula (4)-2 above are similar to n¹, R¹, Ar¹, and Ar² defined in the general formula (3) above. X¹ in the general formula (4)-2 is a halogen atom or a perfluoroalkanesulfonic ester group.

The second method is a method for producing the organic material, comprising reacting a compound represented by the general formula (5)-1 below with a compound represented by the general formula (5)-2 below using a metal catalyst. As the metal catalyst, a palladium catalyst or a copper catalyst may be used.

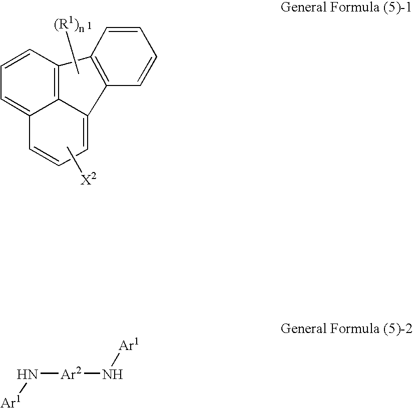

n¹, R¹, Ar¹, and Ar² in the general formula (5)-1 and general formula (5)-2 above are similar to n¹, R¹, Ar¹, and Ar² defined in the general formula (3) above. X² in the general formula (5)-1 is a halogen atom or a perfluoroalkanesulfonic ester group.

The third method is a method for producing the organic material, comprising reacting a compound represented by the general formula (6)-1 below with a compound represented by the general formula (6)-2 below using a metal catalyst. As the metal catalyst, a palladium catalyst or a copper catalyst may be used.

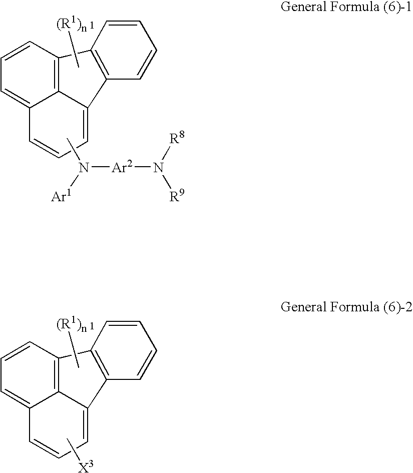

n¹, R¹, Ar¹, and Ar² in the general formula (6)-1 and general formula (6)-2 above are similar to n¹, R¹, Ar¹, and Ar² defined in the general formula (3) above. In the general formula (6)-1, R⁵ is a hydrogen atom or Ar¹, and R⁹ is a hydrogen atom, and X³ in the general formula (6)-2 is a halogen atom or a perfluoroalkanesulfonic ester group.

The fourth method is a method for producing the organic material, comprising reacting a compound represented by the general formula (7) below using an equivalent amount of a metal (e.g., copper), a metal salt (e.g., copper or nickel), or a metal catalyst (e.g., nickel, palladium, or copper).

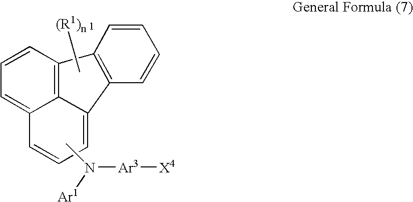

n¹, R¹, and Ar¹ in the general formula (7) above are similar to n¹, R¹, and Ar¹ defined in the general formula (3) above. Ar³ in the general formula (7) is a divalent group which is derived from monocyclic or fused-ring aromatic hydrocarbon having 1 to 3 rings, and which may have a substituent having 4 carbon atoms or less, and X⁴ is a halogen atom or a perfluoroalkanesulfonic ester group.

In the fourth method, the compound represented by the general formula (7) above may be reacted with a compound corresponding to the compound represented by the general formula (7) wherein X⁴ is changed to magnesium halide, boric acid, or borate.

The organic material represented by the general formula (3) is synthesized by any one of the above first to fourth methods.

### BRIEF DESCRIPTION OF THE DRAWING

FIG. 1 is an NMR spectrum of the synthesized compound of the structural formula (2)-o.

### BEST MODE FOR CARRYING OUT THE INVENTION

Hereinbelow, embodiments of the present invention will be described. The first organic light-emitting material of the present invention is characterized in that it is used in a light emitting layer in a green light emitting element (e.g., organic EL element) and represented by the following general formula (1):

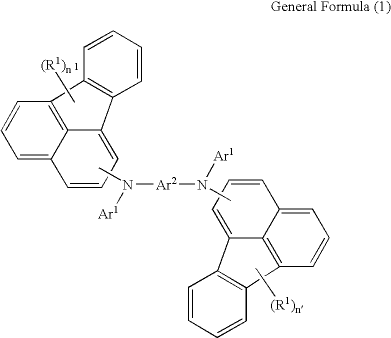

In the general formula (1) above, n¹ is an integer of 0 to 3, and R¹ is an alkyl group having 10 carbon atoms or less. When n¹ is 2 or 3 and each fluoranthene is substituted with two or three R¹'s at a plurality of positions (carbon atoms numbered), each of the R¹'s may be independently an alkyl group having 10 carbon atoms or less. Ar¹ is a monovalent group derived from monocyclic or fused-ring aromatic hydrocarbon having 20 carbon atoms or less, and may have a substituent having 10 carbon atoms or less. Ar² is a divalent group derived from a ring assembly having 30 carbon atoms or less and being comprised of monocyclic or fused-ring aromatic hydrocarbon having 1 to 3 rings. The divalent group may have a substituent having 4 carbon atoms or less.

As a specific example of the organic light-emitting material emitting green light, there can be mentioned a material of the structural formula (1) below corresponding to the general formula (1) wherein Ar¹ is an unsubstituted phenyl group, n¹ is 0, and Ar² is a divalent group derived from unsubstituted biphenyl.

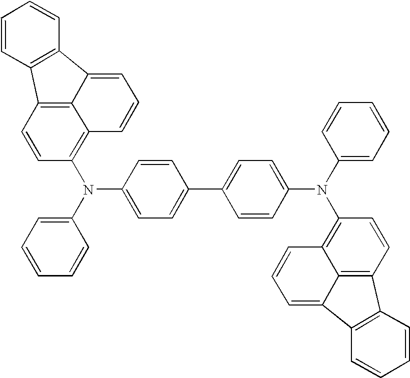

The second organic light-emitting material of the present invention is an organic light-emitting material represented by the following general formula (2):

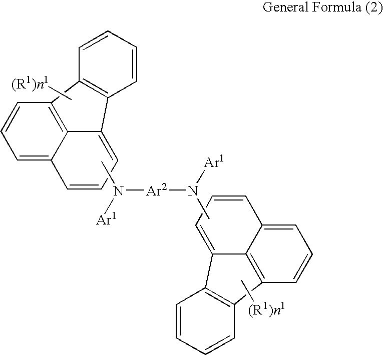

n¹, R¹, Ar¹, and Ar² in the general formula (2) above are similar to those defined in the general formula (1) above. Specifically, n¹ is an integer of 0 to 3, and R¹ is an alkyl group having 10 carbon atoms or less. When n¹ is 2 or 3 and each fluoranthene is substituted with R¹'s at a plurality of positions (carbon atoms numbered), each of the R¹'s may be independently an alkyl group having 10 carbon atoms or less. Ar¹ is a monovalent group derived from monocyclic or fused-ring aromatic hydrocarbon having 20 carbon atoms or less, and may have a substituent having 10 carbon atoms or less. Ar² is a divalent group derived from a ring assembly having 30 carbon atoms or less and being comprised of monocyclic or fused-ring aromatic hydrocarbon having 1 to 3 rings, and may have a substituent having 4 carbon atoms or less. In the general formula (2) above, the case where the monovalent group is an unsubstituted phenyl group, the divalent group is a divalent group derived from unsubstituted biphenyl, and each of two fluoranthenes is bonded to nitrogen at the carbon numbered 3 is excluded. That is, the second organic light-emitting material does not encompass the material of the structural formula (1) above. In contrast, the first organic light-emitting material encompasses the second organic light-emitting material described in detail here.

The organic light-emitting material (second organic light-emitting material) is a light emitting material used in a light emitting layer in a green light emitting element (e.g., organic EL element).

Particularly, as the ring assembly constituting Ar² in the general formula (2), for example, biphenyl, binaphthyl, or bianthracenyl can be used. Ar² may have a substituent having 4 carbon atoms or less in a divalent group derived from the above ring assembly.

With respect to the second organic light-emitting material, as an example of the organic light-emitting material of the general formula (2) wherein the ring assembly constituting Ar² is biphenyl and the monovalent group which is derived from monocyclic or fused-ring aromatic hydrocarbon and which constitutes Ar¹ is a phenyl group, there can be mentioned an organic light-emitting material represented by the following general formula (8):

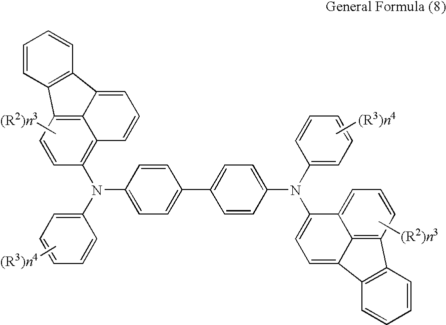

R² and n³ in the general formula (8) above respectively correspond to R¹ and n¹ in the general formula (2) above. R³ in the general formula (8) corresponds to the substituent having 10 carbon atoms or less in Ar¹ in the general formula (2), and n⁴ in the general formula (8) corresponds to the number of the substituent(s) having 10 carbon atoms or less in Ar¹ in the general formula (2). In the general formula (8) above, each of R² multiplied by n³ bonded to the positions in each fluoranthene is independently an alkyl group selected from a methyl group, an ethyl group, an i-propyl group, and a t-butyl group, and n³ is an integer of 0 to 3. Further, in the general formula (8), each of R³ multiplied by n⁴ is independently an alkyl group selected from a methyl group, an ethyl group, an i-propyl group, and a t-butyl group, or a phenyl group, and n⁴ is an integer of 0 to 3. As shown in the general formula (8), when Ar¹ in the general formula (2) is a phenyl group, n⁴ is preferably an integer of 1 to 3.

The organic light-emitting material of the general formula (2) wherein the monovalent group which is derived from monocyclic or fused-ring aromatic hydrocarbon and which constitutes Ar¹ has a substituent having 10 carbon atoms or less and the substituent {e.g., R³ in the general formula (8)} is an alkyl group selected from a methyl group, an ethyl group, an i-propyl group, and a t-butyl group, or a phenyl group is a material having excellent amorphous properties as mentioned below.

As specific examples of the organic light-emitting materials, there can be mentioned compounds of the following structural formulae (2)-p to (3)-o.

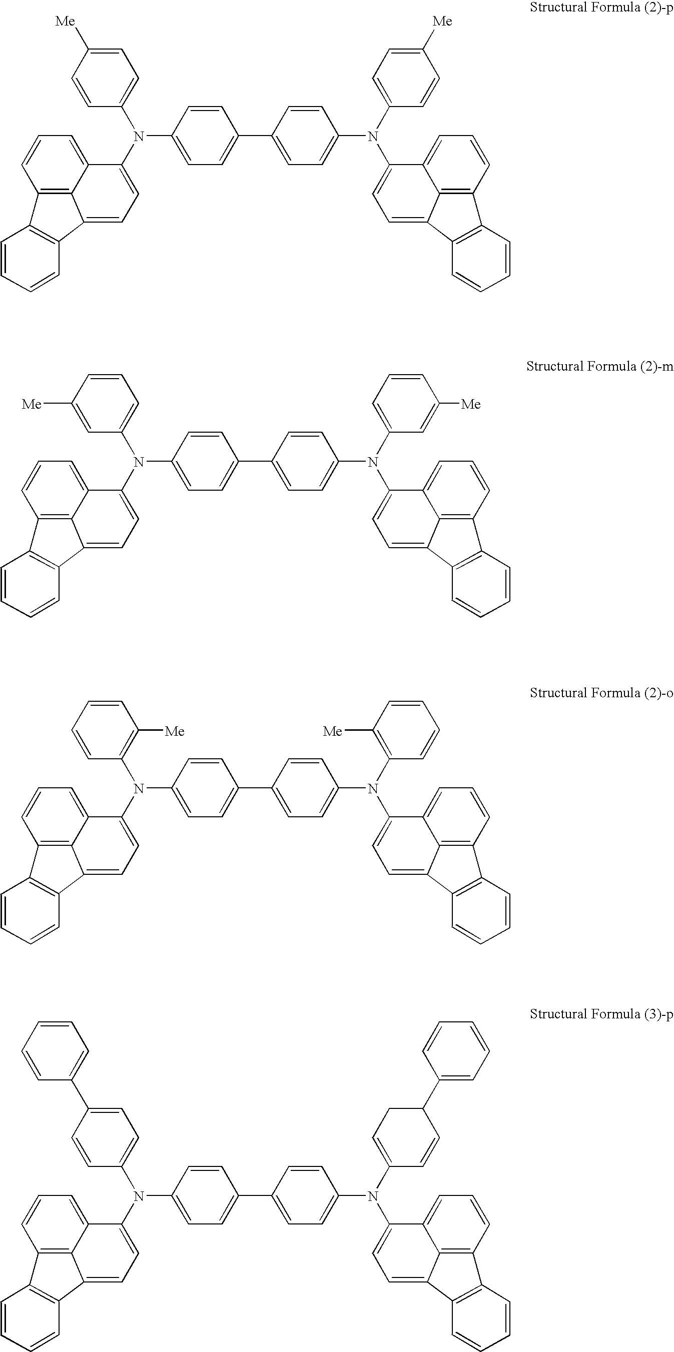

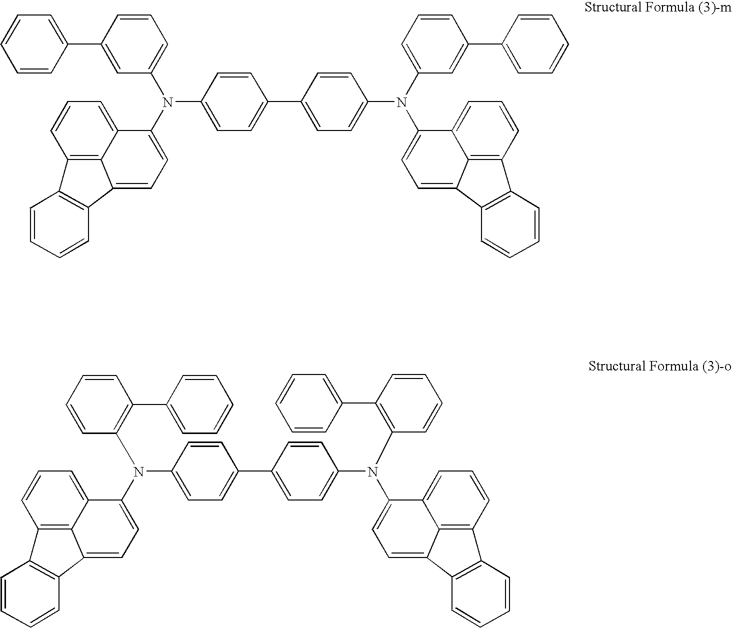

In the second organic light-emitting material, when the ring assembly constituting Ar² in the general formula (2) above is biphenyl, the monovalent group which is derived from monocyclic or fused-ring aromatic hydrocarbon and which constitutes Ar¹ is not limited to a phenyl group. For example, the monovalent group may be a monovalent group derived from fluoranthene or naphthalene. As specific examples of the organic light-emitting materials, there can be mentioned compounds of the following structural formulae (7) and (8).

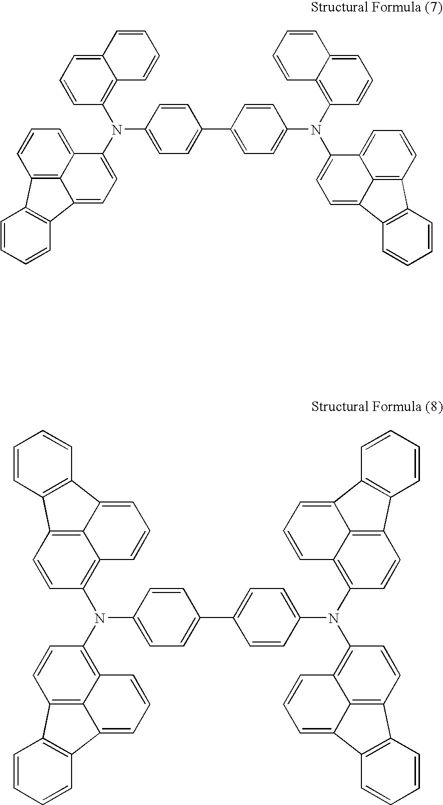

Among these, the organic light-emitting material of the structural formulae (2)-p to (3)-o or structural formula (7) corresponding to the general formula (2) wherein Ar¹ (including a substituent) is a biphenyl group, a phenyl group having a methyl group, or a naphthyl group is a material having excellent amorphous properties as mentioned below.

When the ring assembly constituting Ar² in the general formula (2) is binaphthyl, it is preferred that the organic light-emitting material has a structure represented by the following general formula (9):

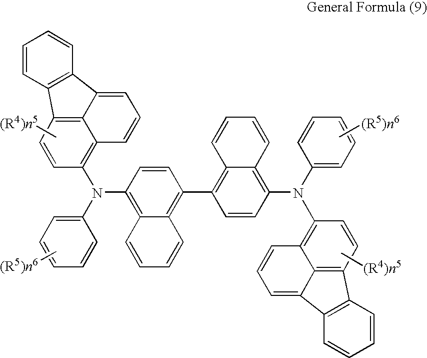

R⁴ and n⁵ in the general formula (9) above respectively correspond to R¹ and n¹ in general formula (2) above. R⁵ in the general formula (9) corresponds to the substituent having 10 carbon atoms or less in Ar¹ in general formula (2), and n⁶ in the general formula (9) corresponds to the number of the substituent(s) having 10 carbon atoms or less in Ar¹ in the general formula (2). In the general formula (9), each of R⁴ multiplied by n⁵ bonded to the positions in each fluoranthene is independently an alkyl group selected from a methyl group, an ethyl group, an i-propyl group, and a t-butyl group, and n⁵ is an integer of 0 to 3. Further, in the general formula (9), each of R⁵ multiplied by n⁶ is independently an alkyl group selected from a methyl group, an ethyl group, an i-propyl group, and a t-butyl group, or a phenyl group, and n⁶ is an integer of 0 to 3.

As a specific example of the organic light-emitting material, there can be mentioned a compound of the structural formula (9) below. Particularly, the organic light-emitting material represented by the structural formula (9) is a material having excellent amorphous properties as mentioned below.

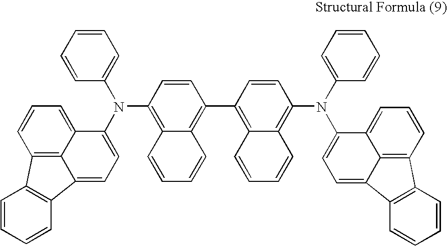

When the ring assembly constituting Ar² in the general formula (2) is bianthracenyl, it is preferred that the organic light-emitting material has a structure represented by the following general formula (10):

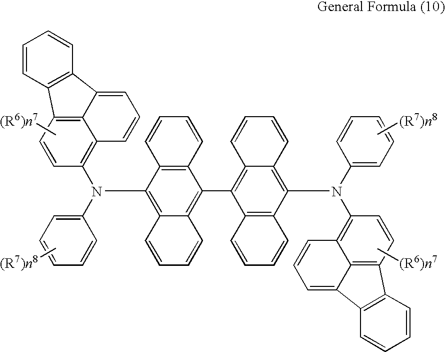

R⁶ and n⁷ in the general formula (10) above respectively correspond to R¹ and n¹ in the general formula (2) above. R⁷ in the general formula (10) corresponds to the substituent having 10 carbon atoms or less in Ar¹ in the general formula (2), and n⁸ in the general formula (10) corresponds to the number of the substituent(s) having 10 carbon atoms or less in Ar¹ in the general formula (2). In the general formula (10), each of R⁶ multiplied by n⁷ bonded to the positions in each fluoranthene is independently an alkyl group selected from a methyl group, an ethyl group, an i-propyl group, and a t-butyl group, and n⁷ is an integer of 0 to 3. Further, in the general formula (10), each of R⁷ multiplied by n⁸ is independently an alkyl group selected from a methyl group, an ethyl group, an i-propyl group, and a t-butyl group, or a phenyl group, and n⁸ is an integer of 0 to 3.

As a specific example of the organic light-emitting material, there can be mentioned a compound of the following structural formula (10).

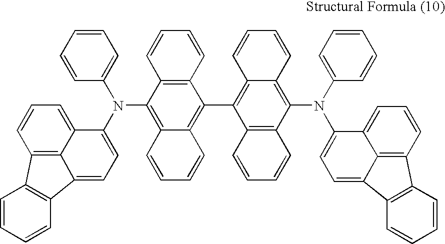

As examples of the second organic light-emitting materials represented by the general formula (2), compounds of the general formulae (8) to (10) and structural formulae (1) to (9) wherein each of two fluoranthenes is bonded to nitrogen at the position of carbon atom numbered 3 are shown. However, the second organic light-emitting material is not limited to these, and may be a compound in which each of two fluoranthenes is bonded to nitrogen at another position, for example, as shown in the structural formula (11) below. Particularly, the organic light-emitting material represented by the structural formula (11) is a material having excellent amorphous properties as mentioned below.

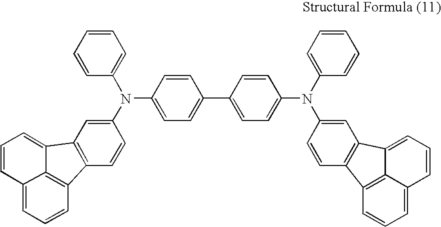

When each fluoranthene is bonded to nitrogen at another position {carbon numbered 8 in the structural formula (11)}, n¹, R¹, Ar¹, and Ar² in the general formula (2) are similar to those described above using the general formulae (8) to (10).

Each of the first and second organic light-emitting materials described above is used as a material constituting a light emitting layer in an organic element, and particularly used as a guest material having light emitting properties in a light emitting layer in a green light emitting organic element. Thus, a green light emitting organic element having excellent chromaticity can be obtained.

Particularly, each of the above-described first organic light-emitting material and second organic light-emitting material of the present invention has a very strong molecular skeleton comprised of 3 constituent elements. In other words, Alq3 conventionally widely used as an organic light-emitting material emitting green light is comprised of 5 constituent elements (carbon, hydrogen, oxygen, nitrogen, and aluminum). In addition, many conventional organic light-emitting materials emitting green light including coumarin and quinacridone are comprised of 4 constituent elements or more. The number of constituent elements of the organic light-emitting material of the present invention is 3, which is small, as compared to that of the conventional organic light-emitting material emitting green light, and thus a stronger molecular skeleton is achieved. Therefore, the organic light-emitting material of the present invention has such a high resistance as an organic light-emitting material emitting green light that it is prevented from deteriorating. In addition, when the organic light-emitting material is used in a green light emitting layer, an organic light-emitting element having high chromaticity and high luminance is formed.

Next, a method for producing the first EL light emitting material represented by the general formula (1) above and a method for producing the second organic light-emitting material represented by the general formula (2) above will be described. The organic material obtained by the method described below is not limited to a material used as an organic light-emitting material.

First, the first method for obtaining the organic material is a method which comprises reacting a compound represented by (4)-1 with a compound represented by the general formula (4)-2 using a metal catalyst. As the metal catalyst, a palladium catalyst or a copper catalyst may be used.

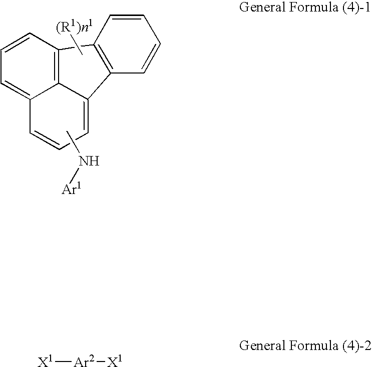

n¹, R¹, Ar¹, and Ar² in the general formula (4)-1 and general formula (4)-2 above are similar to n¹, R¹, Ar¹, and Ar² defined in the general formulae used in the above descriptions of the first organic light-emitting material and the second organic light-emitting material. X¹ in the general formula (4)-2 is a halogen atom or a perfluoroalkanesulfonic ester group. When X¹ is a halogen atom, bromine or iodine is used.

Particularly, in the first method, as the ring assembly constituting Ar² in the general formula (4)-2, for example, biphenyl, binaphthyl, or bianthracenyl is preferably used.

The second method for obtaining the organic material is a method which comprises reacting a compound represented by the general formula (5)-1 below with a compound represented by the general formula (5)-2 below using a metal catalyst. As the metal catalyst, a palladium catalyst or a copper catalyst may be used.

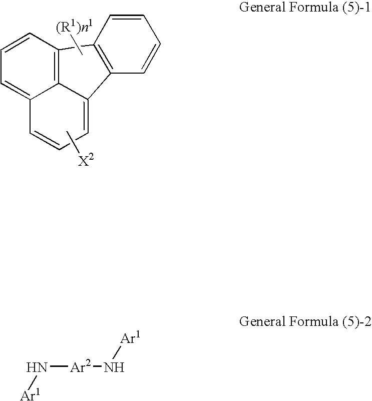

n¹, R¹, Ar¹, and Ar² in the general formula (5)-1 and general formula (5)-2 above are similar to n¹, R¹, Ar¹, and Ar² defined in the general formulae used in the above descriptions of the first organic light-emitting material and the second organic light-emitting material. X² in the general formula (5)-1 is a halogen atom or a perfluoroalkanesulfonic ester group. When X² is a halogen atom, bromine or iodine is used.

Particularly, in the second method, as the ring assembly constituting Ar² in the general formula (5)-2, for example, biphenyl, binaphthyl, or bianthracenyl is preferably used.

The third method is a method which comprises reacting a compound represented by the (6)-1 below with a compound represented by the general formula (6)-2 below using a metal catalyst. As the metal catalyst, a palladium catalyst or a copper catalyst may be used.

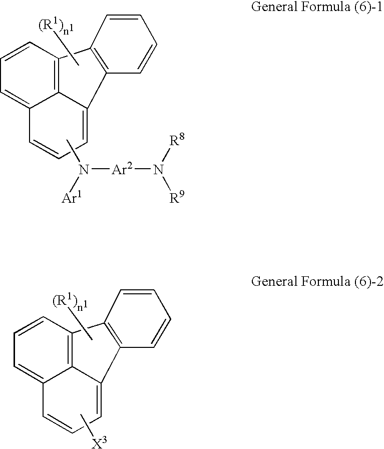

n¹, R¹, Ar¹, and Ar² in the general formula (6)-1 and general formula (6)-2 above are similar to n¹, R¹, Ar¹, and Ar² defined in the general formulae used in the above descriptions of the first organic light-emitting material and the second organic light-emitting material. In the general formula (6)-1, R⁵ is a hydrogen atom or Ar¹, and R⁹ is a hydrogen atom, and X³ in the general formula (6)-2 is a halogen atom or a perfluoroalkanesulfonic ester group. When X³ is a halogen atom, bromine or iodine is used.

Particularly, in the third method, as the ring assembly constituting Ar² in the general formula (6)-1, for example, biphenyl, binaphthyl, or bianthracenyl is preferably used.

The fourth method is a method which comprises reacting a compound represented by the general formula (7) below using an equivalent amount of a metal (copper), a metal (copper or nickel) salt, or a metal catalyst (a nickel catalyst, a palladium catalyst, or a copper catalyst).

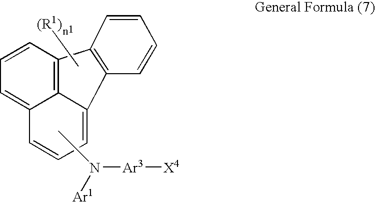

n¹, R¹, and Ar¹ in the general formula (7) above are similar to n¹, R¹, and Ar¹ defined in the general formulae used in the above descriptions of the first organic light-emitting material and the second organic light-emitting material. In the general formula (7), Ar³ is a divalent group which is derived from monocyclic or fused-ring aromatic hydrocarbon having 1 to 3 rings, and which may have a substituent having 4 carbon atoms or less, and X⁴ is a halogen atom or a perfluoroalkanesulfonic ester group. When X⁴ is a halogen atom, bromine or iodine is used.

Particularly, in the fourth method, as the ring assembly constituting Ar³ in the general formula (7), for example, a divalent group derived from benzene, naphthalene, or anthracene is preferably used.

Further, in the fourth method, the compound represented by the general formula (7) above may be reacted with a compound corresponding to the compound represented by the general formula (7) wherein X³ is changed to magnesium halide, boric acid, or borate.

### EXAMPLES

Hereinbelow, Examples of the present invention will be described. A method of synthesizing an organic light-emitting material by the second method described above using the general formula (5)-1 and general formula (5)-2 is described below.

### Example 1

A compound of the structural formula (1) was synthesized as follows.

3-Bromofluoranthene (9.0 g, 32 mmol) was first added in three portions to a mixture of toluene (200 ml), tri(t-butyl)phosphine (0.4 g, 20 mmol), palladium acetate (0.1 g, 4.5 mmol), N,N-diphenylbenzidine (4.8 g, 14 mmol), and sodium t-butoxide (4.8 g, 50 mmol), and reacted by heating at 90° C. for 50 hours.

The resultant reaction mixture was cooled to room temperature, and then crystals were collected by filtration and washed with a small amount of toluene. The crude product was purified by silica gel chromatography, and the resultant product was purified by sublimation to obtain a compound (3.5 g; 34%) of the structural formula (1).

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=736.4 [(M⁺)]

(b) ¹H-NMR (400 MHz, CDCl₃); 7.00 (m, 2H), 7.10-7.18 (8H), 7.20-7.28 (4H), 7.30-7.47 (12H), 7.65 (d, 2H, J=8.5 Hz), 7.70-7.80 (8H)

(c) UV-VIS absorption spectrum peak 443 nm

(d) Fluorescence spectrum peak 543 nm (in dioxane)

The results of the analyses of the items (a) and (b) above confirm that the compound of the structural formula (1) was synthesized by the synthesis method in the present Example. Further, the peaks of the fluorescence spectrum of the item (d) above confirm that the film of the synthesized compound of the structural formula (1) emits green light with excellent chromaticity.

### Example 2

A compound of the structural formula (2)-p was synthesized in accordance with the following reaction scheme (1).

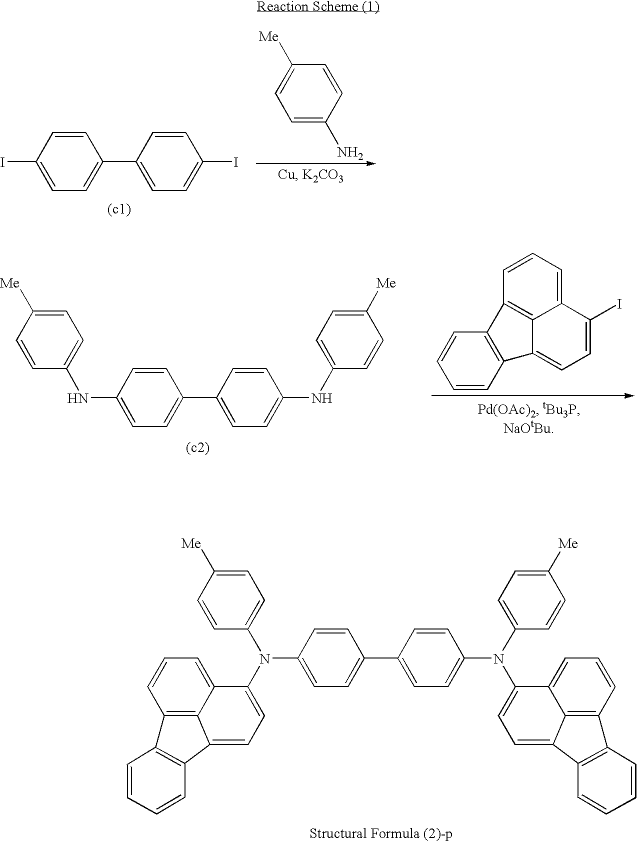

(c1) 4,4′-Diiodo-1,1′-biphenyl (35 g, 86 mmol), 4-methylaniline (92 g, 86 mmol), copper powder (2.7 g, 43 mmol), and potassium carbonate (12 g, 86 mmol) were first stirred at 170° C. for 24 hours. Tetrahydrofuran (400 ml) was added to the reactor and the resultant mixture was filtered, and the filtrate was subjected to vacuum evaporation. The resultant residue was washed successively with ethyl acetate, n-hexane, and acetonitrile, and then the resultant crystals were dried to obtain (c2) N,N′-bis(4-methylphenyl)benzidine (13 g; 40%).

Next, (c2) N,N′-bis(4-methylphenyl)benzidine (11 g, 28 mmol) was added in three portions to a mixture of 3-iodofluoranthene (20 g, 70 mmol), palladium acetate (0.2 g, 0.89 mmol), tri-t-butylphosphine (0.6 g, 3.0 mmol), sodium t-butoxide (7.9 g, 82 mmol), and dried toluene (370 ml), and stirred at 110° C. for 18 hours. The resultant reaction mixture was cooled to room temperature and filtered, and the filtrate was subjected to vacuum evaporation. The resultant residue was purified by silica gel chromatography to obtain difluoranthenyl (8.8 g; 42%) of the structural formula (2)-p.

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=763.7 [(M⁺)]

(b) ¹H-NMR (CDCl₃) δ (ppm); 2.16 (s, 6H), 7.06 (s, 10H), 7.08 (m, 2H), 7.29-7.43 (12H), 7.65 (d, 2H, J=6.5 Hz), 7.80-7.89 (8H)

(c) UV-VIS absorption spectrum peak 451 nm

(d) Fluorescence spectrum peak 551 nm (in dioxane)

The results of the analyses of the items (a) and (b) above confirm that the compound of the structural formula (2)-p was synthesized by the synthesis method in the present Example. Further, the peaks of the fluorescence spectrum of the item (d) above confirm that the film of the synthesized compound of the structural formula (2)-p emits green light with excellent chromaticity.

### Example 3

A compound of the structural formula (2)-m was synthesized in accordance
with the following reaction scheme (2).

[symbol]

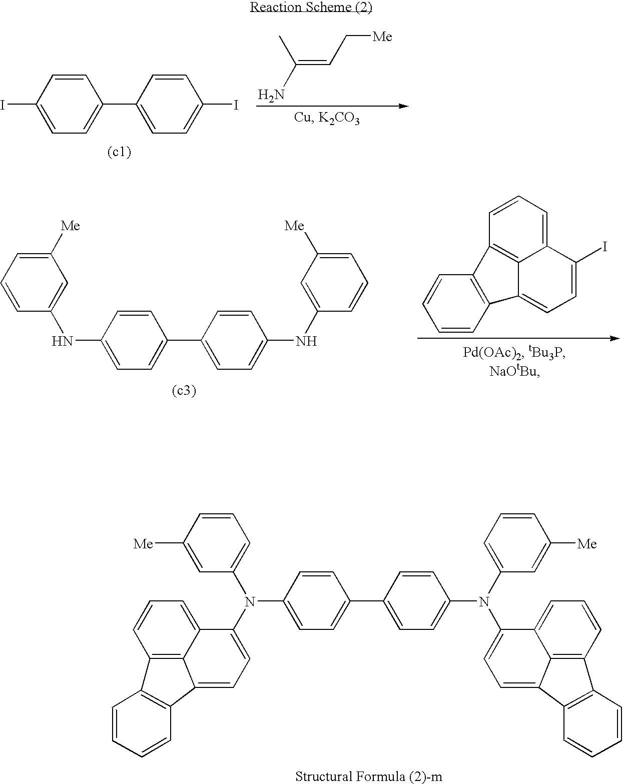

(c1) 4,4′-Diiodo-1,1′-biphenyl (20 g, 49 mmol), 3-methylaniline (195 g, 1.8 mol), copper powder (11 g, 160 mmol), and potassium carbonate (25 g, 180 mmol) were first heated at 170° C. for 24 hours. The reactor was cooled, and the resultant solids were collected by filtration and washed successively with xylene and ethyl acetate. Tetrahydrofuran (400 ml) was added to the solids and the resultant mixture was filtered, and the filtrate was subjected to vacuum evaporation. The resultant residue was subjected to recrystallization from tetrahydrofuran-methanol, and subjected to slurry washing twice using acetonitrile to obtain (c3) N,N′-bis(3-methylphenyl)benzidine (3.1 g; 17%).

Next, (c3) N,N′-bis(3-methylphenyl)benzidine (3.0 g, 8.2 mmol) was added in three portions to a mixture of 3-iodofluoranthene (5.9 g, 18 mmol), palladium acetate (55 mg, 0.25 mmol), tri-t-butylphosphine (0.2 ml, 0.82 mmol), sodium t-butoxide (2.4 g, 25 mmol), and dried toluene (100 ml), and stirred at 100° C. for 17 hours. The reactor was cooled, and tetrahydrofuran (450 ml) was added to the reactor and the resultant mixture was filtered, and the filtrate was subjected to vacuum evaporation. The resultant crystals were subjected to recrystallization from xylene to obtain a difluoranthenyl compound (3.0 g; 48%) of the structural formula (2)-m.

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=763.7 [(M⁺)]

(b) ¹H-NMR (CDCl₃) δ (ppm); 2.13 (s, 6H), 6.82 (m, 2H), 6.92-6.98 (4H), 7.08-7.15 (6H), 7.31-7.45 (12H), 7.65 (d, 2H, J=8 Hz), 7.81-7.80 (8H)

(c) UV-VIS absorption spectrum peak 448 nm

(d) Fluorescence spectrum peak 546 nm (in dioxane)

The results of the analyses of the items (a) and (b) above confirm that the compound of the structural formula (2)-m was synthesized by the synthesis method in the present Example. Further, the peaks of the fluorescence spectrum of the item (d) above confirm that the film of the synthesized compound of the structural formula (2)-m emits green light with excellent chromaticity.

### Example 4

A compound of the structural formula (2)-o was synthesized in accordance with the following reaction scheme (3).

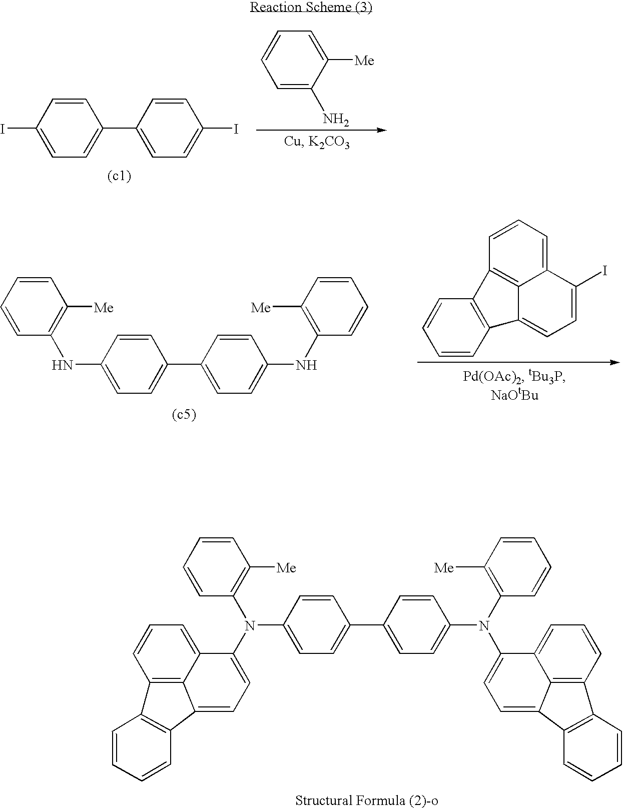

(c1) 4,4′-Diiodo-1,1′-biphenyl (19 g, 47 mmol), 2-methylaniline (180 g, 1.7 mol), copper powder (10 g, 160 mmol), and potassium carbonate (23 g, 170 mmol) were first heated at 170° C. for 23 hours. The reactor was cooled, and the resultant solids were collected by filtration and washed with tetrahydrofuran. The resultant washing liquid was subjected to vacuum evaporation to obtain crude crystals. The crude crystals were subjected to recrystallization from tetrahydrofuran-methanol to obtain (c5) N,N′-bis(2-methylphenyl)benzidine (11 g; 64%).

(c5) N,N′-Bis(2-methylphenyl)benzidine (9.2 g, 25 mmol) was added in three portions to a mixture of 3-iodofluoranthene (18 g, 55 mmol), palladium acetate (170 mg, 0.76 mmol), tri-t-butylphosphine (0.51 g, 2.5 mmol), sodium t-butoxide (7.2 g, 75 mmol), and dried xylene (370 ml), and stirred at 100° C. for 17 hours. The reactor was cooled, and tetrahydrofuran was added to the reactor and the resultant mixture was filtered. The filtrate was concentrated and the resultant crystals were subjected to slurry washing using methanol, and subjected to recrystallization from xylene four times to obtain a difluoranthenyl compound (6.2 g; 32%) represented by the structural formula (2)-o.

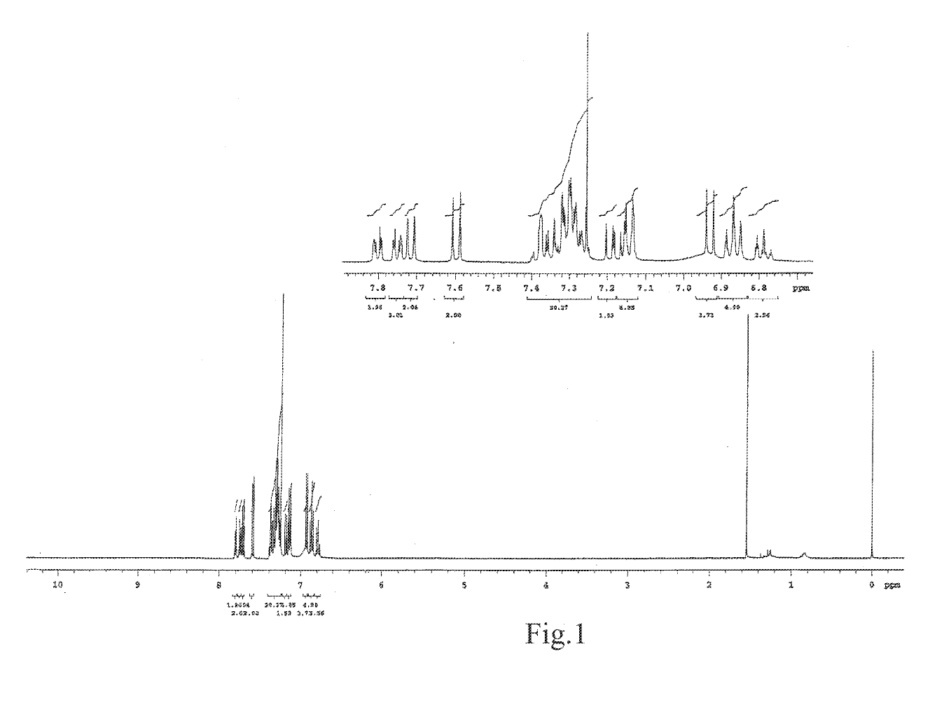

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained. An NMR spectrum of the above-obtained compound of the structural formula (2)-o is shown in FIG. 1.

(a) MS [TOF] m/z=763.3 [(M⁺)]

(b) ¹H-NMR (CDCl₃, 400 MHz) δ (ppm); 2.09 (s, 6H), 6.91 (m, 2H), 7.09 (dt, 2H, J=7 Hz, 7 Hz), 7.10-7.19 (6H), 7.22-7.28 (4H), 7.29-7.44 (10H), 7.61 (d, 2H, J=8 Hz), 7.75 (d, 2H, J=8 Hz), 7.78-7.88 (6H)

(c) UV-VIS absorption spectrum peak 455 nm

(d) Fluorescence spectrum peak 536 nm (in dioxane)

The results of the analyses of the items (a) and (b) above confirm that the compound of the structural formula (2)-o was synthesized by the synthesis method in the present Example. Further, the peaks of the fluorescence spectrum of the item (d) above confirm that the film of the synthesized compound of the structural formula (11) emits green light with excellent chromaticity.

### Example 5

A compound of the structural formula (3)-p was synthesized in accordance with the following reaction scheme (4).

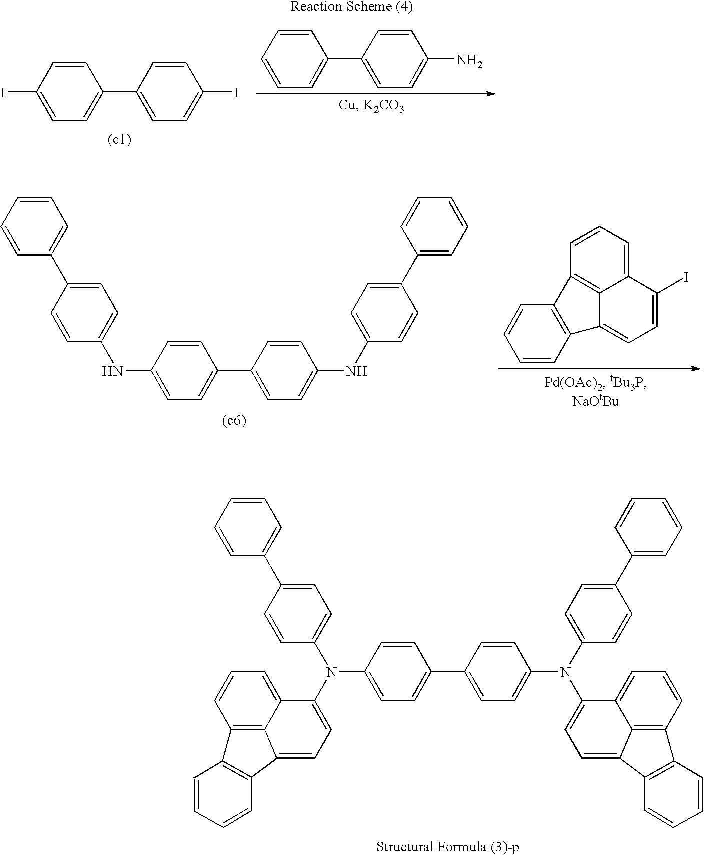

(c1) 4,4′-Diiodo-1,1′-biphenyl (9.0 g, 23 mmol), 4-aminobiphenyl (38 g, 230 mmol), copper powder (6.9 g, 110 mmol), and potassium carbonate (15 g, 110 mmol) were first heated at 100° C. for 15 hours. The reactor was cooled, and the resultant solids were collected by filtration and washed successively with xylene and ethyl acetate. Tetrahydrofuran (400 ml) was added to the solids, and the resultant mixture was filtered and the solvent was removed by vacuum evaporation. The resultant residue was subjected to slurry washing using hot xylene to obtain (c6) N,N′-bis(4-biphenylyl)benzidine (5.5 g; 49%).

(c6) N,N′-Bis(4-biphenylyl)benzidine (10 g, 20 mmol) was added in three portions to a mixture of 3-iodofluoranthene (4.5 g, 9.2 mmol), palladium acetate (60 mg, 0.27 mmol), tri-t-butylphosphine (0.18 g, 0.89 mmol), sodium t-butoxide (2.7 g, 29 mmol), and dried xylene (200 ml), and stirred at 110° C. for 12 hours. The reactor was cooled, followed by filtration. The resultant solids were washed successively with xylene and ethyl acetate, and then extracted with tetrahydrofuran. The filtrate was concentrated, and the resultant solids were subjected to slurry washing using ethyl acetate and hot xylene to obtain a difluoranthenyl compound (5.3 g; 30%) represented by the structural formula (3)-p.

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=887.9 [(M⁺)]

(b) ¹H-NMR (CDCl₃, 400 MHz) δ (ppm) 7.15 (m, 2H), 7.19-7.51 (32H), 7.65 (d, 2H, J=8 Hz), 7.83-7.90 (8H)

(c) UV-VIS absorption spectrum peak 450 nm

(d) Fluorescence spectrum peak 546 nm (in dioxane)

The results of the analyses of the items (a) and (b) above confirm that the compound of the structural formula (3)-p was synthesized by the synthesis method in the present Example. Further, the peaks of the fluorescence spectrum of the item (d) above confirm that the film of the synthesized compound of the structural formula (11) emits green light with excellent chromaticity.

### Example 6

A compound of the structural formula (3)-m was synthesized in accordance with the following reaction scheme (5).

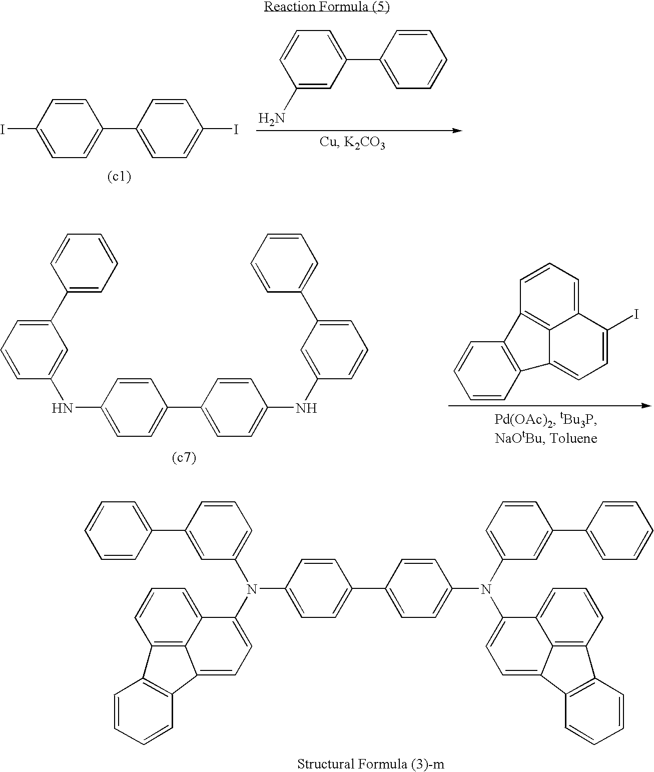

(c1) 4,4′-Diiodo-1,1′-biphenyl (6.0 g, 15 mmol), 3-aminobiphenyl (25 g, 150 mmol), copper powder (4.6 g, 73 mmol), and potassium carbonate (10 g, 73 mmol) were first heated at 100° C. for 20 hours. The reactor was cooled, and the resultant solids were collected by filtration and washed successively with xylene and ethyl acetate. Tetrahydrofuran (400 ml) was added to the solids and the resultant mixture was filtered, and the filtrate was subjected to vacuum evaporation. The resultant residue was subjected to slurry washing using hot xylene to obtain (c7) N,N′-bis(3-biphenylyl)benzidine (3.0 g; 41%).

Next, (c7) N,N′-bis(3-biphenylyl)benzidine (3.0 g, 6.1 mmol) was added in three portions to a mixture of 3-iodofluoranthene (4.4 g, 13 mmol), palladium acetate (40 mg, 0.18 mmol), tri-t-butylphosphine (0.12 g, 0.59 mmol), sodium t-butoxide (1.8 g, 19 mmol), and dried xylene (110 ml), and stirred at 110° C. for 20 hours. The reactor was cooled, followed by filtration. The resultant solids were washed successively with xylene and ethyl acetate, and then extracted with tetrahydrofuran. The extract was concentrated, and the resultant solids were subjected to slurry washing using ethyl acetate and hot xylene to obtain a difluoranthenyl compound (1.4 g; 26%) of the structural formula (3)-m.

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=887.2 [(M⁺)]

(b) ¹H-NMR (CDCl₃) δ (ppm); 7.09 (ddd, 2H, J=1 Hz, 2 Hz, 8 Hz), 7.19 (dt, 4H, J=2 Hz, 8 Hz), 7.23 (dt, 2H, J=2 Hz, 8 Hz), 7.26-7.49 (26H), 7.69 (d, 2H, J=8 Hz), 7.83-7.90 (8H)

(c) UV-VIS absorption spectrum peak 443 nm

(d) Fluorescence spectrum peak 541 nm (in dioxane)

The results of the analyses of the items (a) and (b) above confirm that the compound of the structural formula (3)-m was synthesized by the synthesis method in the present Example. Further, the peaks of the fluorescence spectrum of the item (d) above confirm that the film of the synthesized compound of the structural formula (3)-m emits green light with excellent chromaticity.

### Example 7

A compound of the structural formula (3)-o was synthesized in accordance with the following reaction scheme (6).

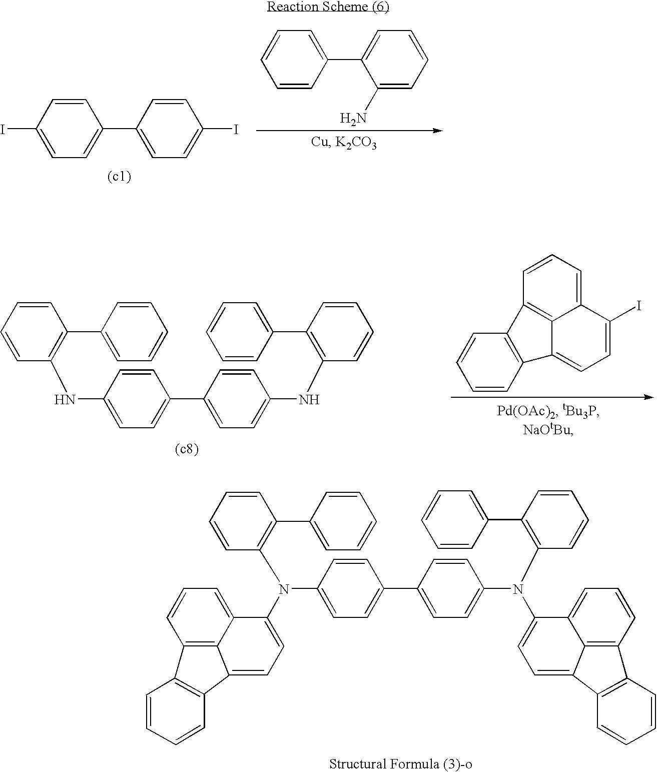

(c1) 4,4′-Diiodo-1,1′-biphenyl (11 g, 27 mmol), 2-aminobiphenyl (46 g, 273 mmol), copper powder (12 g, 180 mmol), potassium carbonate (27 g, 200 mmol), and o-dichlorobenzene (200 ml) were first stirred at 170° C. for 45 hours. Tetrahydrofuran (500 ml) was added to the reactor and the resultant mixture was filtered, and the filtrate was subjected to vacuum evaporation. The resultant residue was purified by column chromatography to obtain (c8) N,N′-bis(2-biphenyl)benzidine (3.4 g; 25%).

Next, (c8) N,N′-bis(2-biphenyl)benzidine (2.3 g, 4.7 mmol) was added in three portions to a mixture of 3-iodofluoranthene (3.4 g, 10 mmol), palladium acetate (63 mg, 0.28 mmol), tri-t-butylphosphine (0.2 ml, 0.93 mmol), sodium t-butoxide (2.7 g, 28 mmol), and dried xylene (70 ml), and stirred at 110° C. for 20 hours. The resultant reaction mixture was cooled to room temperature and filtered, and the filtrate was subjected to vacuum evaporation. The resultant residue was purified by silica gel chromatography to obtain a difluoranthenyl compound (1.4 g; 34%) of the structural formula (3)-o.

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=886.4 [(M⁺)]

(b) ¹H-NMR (CDCl₃) δ (ppm); 6.79 (tt, 2H, J=1 Hz, 7 Hz), 6.84-6.89 (6H), 6.93 (d, 4H, J=8 Hz), 7.12-7.21 (8H), 7.24-7.42 (18H), 7.60 (d, 2H, J=8 Hz), 7.75 (m, 2H), 7.80 (m, 2H)

(c) UV-VIS absorption spectrum peak 448 nm

(d) Fluorescence spectrum peak 532 nm (in dioxane)

The results of the analyses of the items (a) and (b) above confirm that the compound of the structural formula (3)-o was synthesized by the synthesis method in the present Example. Further, the peaks of the absorption spectrum of the item (d) above confirm that the film of the synthesized compound of the structural formula (3)-o emits green light with excellent chromaticity.

### Example 8

A compound of the structural formula (7) was synthesized in accordance with the following reaction scheme (7).

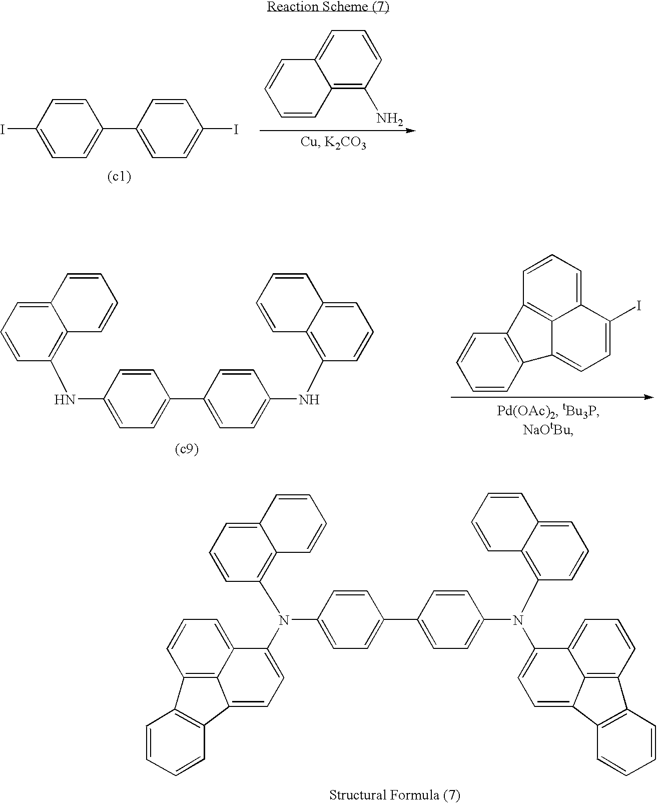

(c1) 4,4′-Diiodo-1,1′-biphenyl (21 g, 52 mmol), 1-aminonaphthalene (75 g, 520 mmol), copper powder (17 g, 260 mmol), potassium carbonate (36 g, 260 mmol), and xylene (1.5 l) were first stirred at 100° C. for 20 hours. The reactor was cooled, and the resultant solids were collected by filtration and washed successively with xylene and ethyl acetate. Tetrahydrofuran (500 ml) was added to the solids and the resultant mixture was filtered, and the filtrate was subjected to vacuum evaporation. The resultant residue was subjected to slurry washing using methanol to obtain (c9) N,N′-bis(1-naphthyl)benzidine (3.0 g; 14%).

Next, (c9) N,N′-bis(1-naphthyl)benzidine (3.0 g, 6.9 mmol) was added in three portions to a mixture of 3-iodofluoranthene (4.9 g, 15 mmol), palladium acetate (50 Mg, 0.22 mmol), tri-t-butylphosphine (0.15 g, 0.74 mmol), sodium t-butoxide (2.0 g, 21 mmol), and dried xylene (120 ml), and stirred at 110° C. for 20 hours. The reactor was cooled, and the resultant solids were collected by filtration and washed successively with xylene and ethyl acetate. The solids were dissolved in xylene while heating, and unnecessary substances were removed by filtration, and the filtrate was subjected to vacuum evaporation. The resultant solids were subjected to slurry washing successively using ethyl acetate and hot xylene to obtain a difluoranthenyl compound (2.1 g; 36%) represented by the structural formula (7).

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=835.8 [(M⁺)]

(b) ¹H-NMR (CDCl₃) δ (ppm); 7.18 (d, 2H, J=7 Hz), 7.25-7.49 (22H), 7.68-7.75 (6H), 7.79 (m, 2H), 7.84-7.92 (6H), 8.06 (m, 2H)

(c) UV-VIS absorption spectrum peak 441 nm

(d) Fluorescence spectrum peak 543 nm (in dioxane)

The results of the analyses of the items (a) and (b) above confirm that the compound of the structural formula (7) was synthesized by the synthesis method in the present Example. Further, the peaks of the fluorescence spectrum of the item (d) above confirm that the film of the synthesized compound of the structural formula (7) emits green light with excellent chromaticity.

### Example 9

A compound of the structural formula (9) was synthesized in accordance with the following reaction scheme (8).

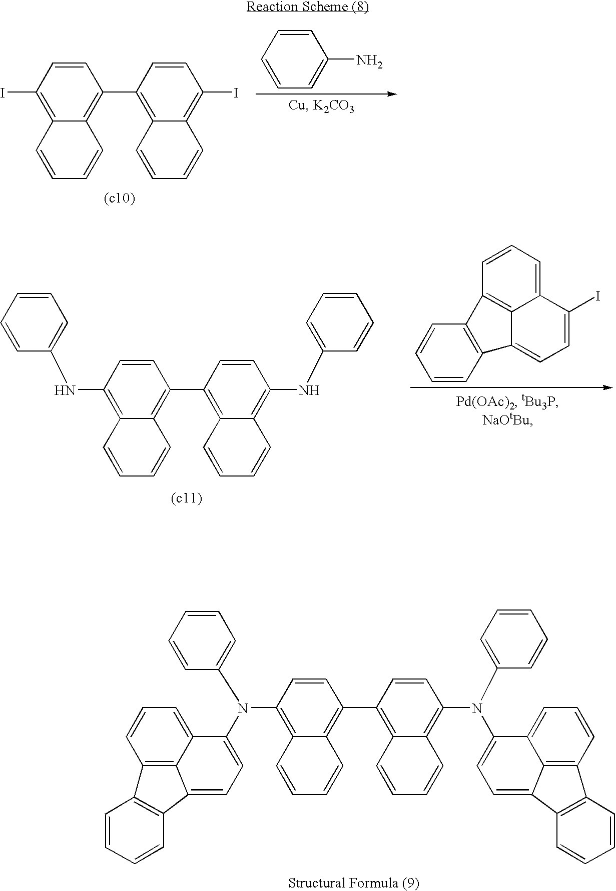

(c10) 4,4′-Diiodo-1,1′-binaphthalene (60 g, 120 mmol), aniline (400 ml), copper powder (23 g, 360 mmol), and potassium carbonate (49 g, 360 mmol) were first heated at 140° C. for 7 hours. The resultant reaction mixture was cooled, and then crystals were removed by filtration and washed with tetrahydrofuran, and the resultant mother liquor was concentrated and purified by silica gel chromatography. The resultant crystals were subjected to slurry washing to obtain (c11) a diphenyl-substituted compound (12 g; 23%).

Next, (c11) the diphenyl-substituted compound (12 g, 28 mmol) was added in three portions to a mixture of 3-iodofluoranthene (20 g, 61 mmol), palladium acetate (0.19 g, 0.85 mmol), tri-t-butylphosphine (0.6 ml, 2.8 mmol), sodium t-butoxide (7.9 g, 82 mmol), and toluene (370 ml), and heated at 100° C. for 5 hours. The resultant reaction mixture was cooled, and then crystals were removed by filtration and washed with tetrahydrofuran, and the resultant mother liquor was concentrated. The resultant residue was purified by silica gel chromatography to obtain a difluoranthenyl compound (8.6 g; 38%) of the structural formula (9).

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=835.7 [(M⁺)]

(b) ¹H-NMR (CDCl₃) δ (ppm); 6.95 (t, 4H, J=7 Hz), 7.11 (d, 8H, J=7 Hz), 7.27-7.36 (12H), 7.41 (d, 4H, J=7 Hz), 7.44-7.54 (12H)

(c) UV-VIS absorption spectrum peak 426 nm

(d) Fluorescence spectrum peak 516 nm (in dioxane)

The results of the analyses of the items (a) and (b) above confirm that the compound of the structural formula (9) was synthesized by the synthesis method in the present Example. Further, the peaks of the fluorescence spectrum of the item (d) above confirm that the film of the synthesized compound of the structural formula (9) emits green light with excellent chromaticity.

### Example 10

A compound of the structural formula (11) was synthesized in accordance with the following reaction scheme (9).

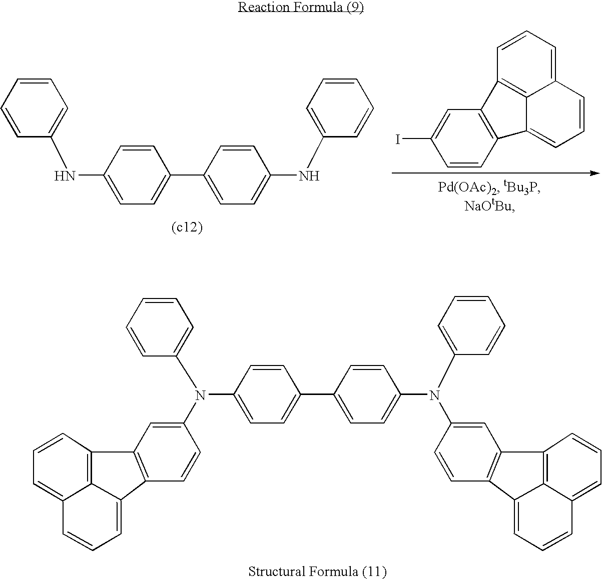

(c12) Diphenylbenzidine (9.3 g, 28 mmol) was first added in three portions to a mixture of 8-iodofluoranthene (20 g, 61 mmol), palladium acetate (0.30 g, 1.3 mmol), tri-t-butylphosphine (1.0 g, 5.0 mmol), sodium t-butoxide (8.1 g, 84 mmol), and toluene (340 ml), and heated at 90° C. for 18 hours. The resultant reaction mixture was cooled, and then crystals were removed by filtration and washed with tetrahydrofuran, and the resultant mother liquor was concentrated. The resultant residue was subjected to slurry washing five times using acetonitrile-tetrahydrofuran to obtain a difluoranthenyl compound (12.2 g; 60%) of the structural formula (11).

With respect to the compound obtained, peaks were measured by (a) mass spectrometric analysis (MS), (b) nuclear magnetic resonance analysis (NMR), (c) ultraviolet-visible absorption spectrum analysis (UV-VIS), and (d) fluorescence spectrum, and the following results were obtained.

(a) MS [TOF] m/z=736.2 [(M⁺)]

(b) ¹H-NMR (CDCl₃) δ (ppm); 7.06 (tt, 2H, J=1 Hz, 8 Hz), 7.14 (dd, 2H, J=2 Hz, 8 Hz), 7.22 (m, 8H), 7.31 (m, 4H), 7.51 (dt, 4H, J=2 Hz, 9 Hz), 7.58 (dd, 2H, J=7 Hz, 8 Hz), 7.61 (dd, 2H, J=7 Hz, 8 Hz), 7.70 (d, 2H, J=2 Hz), 7.79-7.88 (10H)

(c) UV-VIS absorption spectrum peak 433 nm

(d) Fluorescence spectrum peak 535 nm (in dioxane)

The results of the analyses of the items (a) and (b) above confirm that the compound of the structural formula (11) was synthesized by the synthesis method in the present Example. Further, the peaks of the fluorescence spectrum of the item (d) above confirm that the film of the synthesized compound of the structural formula (11) emits green light with excellent chromaticity.

Results of Evaluation

With respect to each of the organic light-emitting materials (difluoranthenyl compounds) synthesized in Examples 1 to 10, a fluorescent quantum yield (in solution) was measured, and a crystallization temperature (Tc) and a glass transition temperature (Tg) were measured by thermal analysis, and the results are shown in the Table 1 below. In the Table 1, a value obtained by subtracting a glass transition temperature (Tg) from a crystallization temperature (Tc) (i.e., Tc−Tg) is shown as a yardstick for the amorphous properties. Further, a chromaticity and a luminance half-life were measured with respect to the organic electroluminescent elements using the individual organic light-emitting materials, and the results are shown in the Table 1. The organic electroluminescent element comprises a light emitting layer comprised of each of the organic light-emitting materials synthesized in Examples 1 to 10 as a guest material and a specific arylanthracene as a host material. With respect to the chromaticity, a value of the organic electroluminescent element having no resonance structure (normal) and a value of the element having a resonance structure (resonance) are shown. The organic electroluminescent element having a resonance structure has a construction such that the thickness of the organic layers including the light emitting layer is controlled to cause the light generated by the light emitting layer to undergo resonance and go outwards.

TABLE 1

| | | Material | | Element | |
|---|---|---|---|---|---|
| | | Fluorescent | Amorphous | Chromaticity | |
| Example | Structural | quantum yield | properties | Normal/ | |
| No. | formula | (In solution) | Tc − Tg | Resonance | Half-life |
| 1 | Structural | 0.77 | 223 − 154 = 69 | (0.358, 0.598)/ | 10,000 h or longer |
| | formula (1) | | | (0.285, 0.677) | |
| 2 | Structural | 0.75 | 223 − 155 = 73 | (0.400, 0.572)/ | About 35,000 h |
| | formula (2)-p | | | (0.359, 0.627) | |
| 3 | Structural | 0.69 | 237 − 146 = 91* | (0.359, 0.604)/ | About 65,000 h* |
| | formula (2)-m | | | (0.290, 0.681) | |
| 4 | Structural | 0.32 | 225 − 162 = 63 | (0.366, 0.595)/ | About 70,000 h* |
| | formula (2)-o | | | (0.259, 0.675)* | |
| 5 | Structural | 0.77 | 238 − 165 = 73 | (0.392, 0.602)/ | About 40,000 h* |
| | formula (3)-p | | | (0.331, 0.642) | |
| 6 | Structural | 0.63 | 273 − 159 = 114* | (0.361, 0.601)/ | About 70,000 h* |
| | formula (3)-m | | | (0.288, 0.622) | |
| 7 | Structural | 0.75 | 230 − 158 = 72 | (0.331, 0.619)/ | About 80,000 h* |
| | formula (3)-o | | | (0.247, 0.695)* | |
| 8 | Structural | 0.59 | 276 − 196 = 80* | (0.358, 0.604)/ | About 70,000 h* |
| | formula (7) | | | (0.264, 0.680)* | |
| 9 | Structural | 0.65 | N.D. − 198 = N.D.* | (0.266, 0.572)/ | About 17,000 h |
| | formula (9) | | | (0.207, 0.662)* | |
| 10 | Structural | 0.61 | 210 − 147 = 63 | (0.329, 0.601)* | About 13,000 h |
| | formula (11) | | | (0.225, 0.674)* | |

Green chromaticity standard: sRGB (0.300, 0.600); NTSC (0.210, 0.710)
*Excellent value for the properties

As can be seen from the Table 1 above, the organic light-emitting materials (difluoranthenyl compounds) synthesized in Examples 1 to 10 have the following effects.

### Example 1

With respect to the organic light-emitting material represented by the structural formula (1) synthesized in Example 1, the fluorescent quantum yield was as high as 0.77. The difference between the crystallization temperature (Tc) and the glass transition temperature (Tg) is as large as 69° C., which confirms that the material has excellent amorphous properties. Further, with respect to the organic electroluminescent element using the material of the structural formula (1) as an organic light-emitting material, the chromaticity in a normal structure is (0.358, 0.598), which indicates that green light emission with high purity close to the sRGB standard could be achieved, and the chromaticity in a resonance structure is (0.285, 0.677), which indicates that green light emission with high purity close to the NTSC standard could be achieved. In addition, it is found that the organic electroluminescent element using the organic light-emitting material represented by the structural formula (1) has an emission lifetime as long as 10,000 hours or longer, in terms of a half-life.

### Example 2

With respect to the organic light-emitting material represented by the structural formula (2)-p synthesized in Example 2, the fluorescent quantum yield was as high as 0.75. The difference between the crystallization temperature (Tc) and the glass transition temperature (Tg) is as large as 73° C., which confirms that the material has excellent amorphous properties. Further, with respect to the organic electroluminescent element using the material of the structural formula (2)-p as an organic light-emitting material, the chromaticity in a normal structure is (0.400, 0.572), which indicates that green light emission with high purity close to the sRGB standard could be achieved, and the chromaticity in a resonance structure is (0.359, 0.627), which indicates that green light emission with high purity close to the NTSC standard could be achieved. In addition, it is found that the organic electroluminescent element using the organic light-emitting material represented by the structural formula (2)-p has an emission lifetime as long as about 35,000 hours, in terms of a half-life.

### Example 3

With respect to the organic light-emitting material represented by the structural formula (2)-m synthesized in Example 3, the fluorescent quantum yield was as high as 0.69. The difference between the crystallization temperature (Tc) and the glass transition temperature (Tg) is as large as 91° C., which confirms that the material has very excellent amorphous properties. Further, with respect to the organic electroluminescent element using the material of the structural formula (2)-m as an organic light-emitting material, the chromaticity in a normal structure is (0.359, 0.604), which indicates that green light emission with high purity close to the sRGB standard could be achieved, and the chromaticity in a resonance structure is (0.290, 0.681), which indicates that green light emission with high purity close to the NTSC standard could be achieved. In addition, it is found that the organic electroluminescent element using the organic light-emitting material represented by the structural formula (2)-m has an emission lifetime as long as about 65,000 hours, in terms of a half-life.

From the above, it is found that, particularly, the organic light-emitting material represented by the structural formula (2)-m has excellent amorphous properties, and that the organic electroluminescent element comprising a light emitting layer using the above organic light-emitting material as a guest material has an improved lifetime.

### Example 4

With respect to the organic light-emitting material represented by the structural formula (2)-o synthesized in Example 4, the fluorescent quantum yield was 0.32. The difference between the crystallization temperature (Tc) and the glass transition temperature (Tg) is as large as 63° C., which confirms that the material has excellent amorphous properties. Further, with respect to the organic electroluminescent element using the material of the structural formula (2)-o as an organic light-emitting material, the chromaticity in a normal structure is (0.366, 0.595), which indicates that green light emission with high purity close to the sRGB standard could be achieved, and the chromaticity in a resonance structure is (0.259, 0.675), which indicates that green light emission with high purity very close to the NTSC standard could be achieved. In addition, it is found that the organic electroluminescent element using the organic light-emitting material represented by the structural formula (2)-o has an emission lifetime as long as about 70,000 hours, in terms of a half-life.

From the above, it is found that, particularly, the organic electroluminescent element comprising a light emitting layer using the organic light-emitting material represented by the structural formula (2)-o as a guest material achieves green light emission with high purity very close to the NTSC standard and has an improved lifetime.

### Example 5

With respect to the organic light-emitting material represented by the structural formula (3)-p synthesized in Example 5, the fluorescent quantum yield was 0.77. The difference between the crystallization temperature (Tc) and the glass transition temperature (Tg) is as large as 73° C., which confirms that the material has excellent amorphous properties. Further, with respect to the organic electroluminescent element using the material of the structural formula (3)-p as an organic light-emitting material, the chromaticity in a normal structure is (0.392, 0.602), which indicates that green light emission with high purity close to the sRGB standard could be achieved, and the chromaticity in a resonance structure is (0.331, 0.642), which indicates that green light emission with high purity close to the NTSC standard could be achieved. In addition, it is found that the organic electroluminescent element using the organic light-emitting material represented by the structural formula (3)-p has an emission lifetime as long as about 40,000 hours, in terms of a half-life.

### Example 6

With respect to the organic light-emitting material represented by the structural formula (3)-m synthesized in Example 6, the fluorescent quantum yield was 0.63. The difference between the crystallization temperature (Tc) and the glass transition temperature (Tg) is as large as 114° C., which confirms that the material has very excellent amorphous properties. Further, with respect to the organic electroluminescent element using the material of the structural formula (3)-m as an organic light-emitting material, the chromaticity in a normal structure is (0.361, 0.601), which indicates that green light emission with high purity close to the sRGB standard could be achieved, and the chromaticity in a resonance structure is (0.288, 0.633), which indicates that green light emission with high purity close to the NTSC standard could be achieved. In addition, it is found that the organic electroluminescent element using the organic light-emitting material represented by the structural formula (3)-m has an emission lifetime as long as about 70,000 hours, in terms of a half-life.

From the above, it is found that, particularly, the organic light-emitting material represented by the structural formula (3)-m has excellent amorphous properties, and that the organic electroluminescent element comprising a light emitting layer using the above organic light-emitting material as a guest material has an improved lifetime.

### Example 7

With respect to the organic light-emitting material represented by the structural formula (3)-o synthesized in Example 7, the fluorescent quantum yield was 0.75. The difference between the crystallization temperature (Tc) and the glass transition temperature (Tg) is as large as 72° C., which confirms that the material has excellent amorphous properties. Further, with respect to the organic electroluminescent element using the material of the structural formula (3)-o as an organic light-emitting material, the chromaticity in a normal structure is (0.331, 0.619), which indicates that green light emission with high purity close to the sRGB standard could be achieved, and the chromaticity in a resonance structure is (0.235, 0.699), which indicates that green light emission with high purity very close to the NTSC standard could be achieved. In addition, it is found that the organic electroluminescent element using the organic light-emitting material represented by the structural formula (3)-o has an emission lifetime as long as about 80,000 hours, in terms of a half-life.

From the above, it is found that, particularly, the organic electroluminescent element comprising a light emitting layer using the organic light-emitting material represented by the structural formula (3)-o as a guest material achieves green light emission with high purity very close to the NTSC standard and has an improved lifetime.

### Example 8

With respect to the organic light-emitting material represented by the structural formula (7) synthesized in Example 8, the fluorescent quantum yield was 0.59. The difference between the crystallization temperature (Tc) and the glass transition temperature (Tg) is as large as 80° C., which confirms that the material has very excellent amorphous properties. Further, with respect to the organic electroluminescent element using the material of the structural formula (7) as an organic light-emitting material, the chromaticity in a normal structure is (0.358, 0.604), which indicates that green light emission with high purity close to the sRGB standard could be achieved, and the chromaticity in a resonance structure is (0.265, 0.680), which indicates that green light emission with high purity very close to the NTSC standard could be achieved. In addition, it is found that the organic electroluminescent element using the organic light-emitting material represented by the structural formula (7) has an emission lifetime as long as about 70,000 hours, in terms of a half-life.

From the above, it is found that, particularly, the organic light-emitting material represented by the structural formula (7) has excellent amorphous properties, and that the organic electroluminescent element comprising a light emitting layer using the above organic light-emitting material as a guest material achieves green light emission with high purity very close to the NTSC standard and has an improved lifetime.

### Example 9

With respect to the organic light-emitting material represented by the structural formula (9) synthesized in Example 9, the fluorescent quantum yield was 0.65. No crystallization temperature (Tc) was detected during the thermal analysis (N.D.), which confirms that the material has very excellent amorphous properties. Further, with respect to the organic electroluminescent element using the material of the structural formula (9) as an organic light-emitting material, the chromaticity in a normal structure is (0.266, 0.572), which indicates that green light emission with high purity close to the sRGB standard could be achieved, and the chromaticity in a resonance structure is (0.207, 0.662), which indicates that green light emission with high purity very close to the NTSC standard could be achieved. In addition, it is found that the organic electroluminescent element using the organic light-emitting material represented by the structural formula (9) has an emission lifetime as long as about 17,000 hours, in terms of a half-life.

From the above, it is found that, particularly, the organic light-emitting material represented by the structural formula (9) has excellent amorphous properties, and that the organic electroluminescent element comprising a light emitting layer using the above organic light-emitting material as a guest material achieves green light emission with high purity very close to the NTSC standard.

### Example 10

With respect to the organic light-emitting material represented by the structural formula (11) synthesized in Example 10, the fluorescent quantum yield was 0.61. The difference between the crystallization temperature (Tc) and the glass transition temperature (Tg) is as large as 63° C., which confirms that the material has excellent amorphous properties. Further, with respect to the organic electroluminescent element using the material of the structural formula (11) as an organic light-emitting material, the chromaticity in a normal structure is (0.329, 0.601), which indicates that green light emission with high purity very close to the sRGB standard could be achieved, and the chromaticity in a resonance structure is (0.225, 0.674), which indicates that green light emission with high purity very close to the NTSC standard could be achieved. In addition, it is found that the organic electroluminescent element using the organic light-emitting material represented by the structural formula (11) has an emission lifetime as long as about 13,000 hours, in terms of a half-life.

From the above, it is found that, particularly, the organic electroluminescent element comprising a light emitting layer using the organic light-emitting material represented by the structural formula (11) as a guest material achieves green light emission with high purity very close to both the sRGB standard and the NTSC standard.

### INDUSTRIAL APPLICABILITY

The above-described first organic light-emitting material and second organic light-emitting material of the present invention can achieve a green light emitting organic element which is advantageous not only in that it has such a high resistance that it is prevented from deteriorating, but also in that it has satisfactorily excellent light emission efficiency and high color purity. Therefore, an organic element using the organic light-emitting material in its organic layer, a red light emitting element, and a blue light emitting element are used in combination to constitute a pixel, enabling full color display with high color reproduction.

By the method for producing an organic material of the present invention, an organic material advantageously used as a material constituting the above green light emitting layer can be synthesized.

1-16. (canceled)

17. An organic light-emitting material comprising a material used in a light emitting layer in a green light emitting element and represented by a following general formula (1): 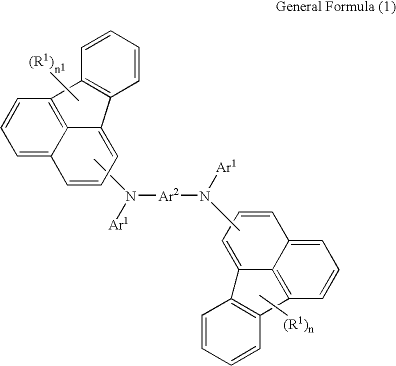
wherein:
n¹ is an integer of 0 to 3;
R¹ is an alkyl group having 10 carbon atoms or less;
Ar¹ is a monovalent group which is derived from monocyclic or fused-ring aromatic hydrocarbon having 20 carbon atoms or less, and which optionally has a substituent having 10 carbon atoms or less; and
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less.

18. The organic light-emitting material according to claim 17, wherein, in the general formula (1) Ar¹ is an unsubstituted phenyl group, n¹ is 0, and Ar² is a divalent group derived from unsubstituted biphenyl.
An organic light-emitting material comprising a material represented by a following general formula (2): 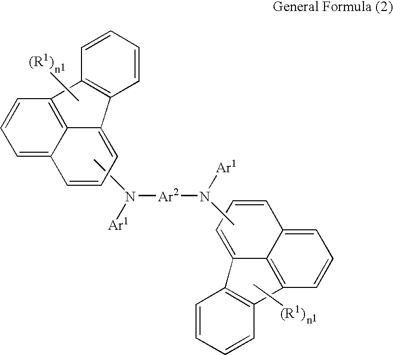
wherein:
n¹ is an integer of 0 to 3;
R¹ is an alkyl group having 10 carbon atoms or less;
Ar¹ is a monovalent group which is derived from monocyclic or fused-ring aromatic hydrocarbon having 20 carbon atoms or less, and which optionally has a substituent having 10 carbon atoms or less; and
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less,
wherein said monovalent group is an unsubstituted phenyl group, said divalent group is a divalent group derived from unsubstituted biphenyl, and each of two fluoranthenes is bonded to nitrogen at the carbon numbered 3 is excluded.

19. The organic light-emitting material according to claim 18, is a light emitting material used in a light emitting layer in a green light emitting organic element.

20. The organic light-emitting material according to claim 18, wherein the ring assembly constituting Ar² in the general formula (2) is biphenyl, binaphthyl, or bianthracenyl.

21. The organic light-emitting material according to claim 18, wherein the monovalent group, which is derived from monocyclic or fused-ring aromatic hydrocarbon, constituting Ar¹ in the general formula (2) has a substituent having 10 carbon atoms or less.

22. The organic light-emitting material according to claim 21, wherein said substituent having 10 carbon atoms or less is an alkyl group selected from the group consisting of a methyl group, an ethyl group, an i-propyl group, and a t-butyl group, and a phenyl group.

23. A method for producing an organic material represented by a general formula (3), the method comprising reacting a compound represented by a general formula (4)-1 with a compound represented by a general formula (4)-2 using a metal catalyst, wherein the general formulas (3), (4)-1 and (4)-2 are as follows: 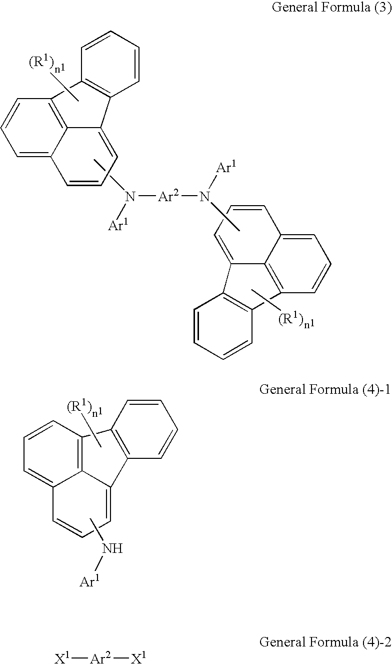
wherein:
in the general formula (3) and general formula (4)-1,
n¹ is an integer of 0 to 3;
R¹ is an alkyl group having 10 carbon atoms or less; and
Ar¹ is a monovalent group which is derived from monocyclic or fused-ring aromatic hydrocarbon having 20 carbon atoms or less, and which optionally has a substituent having 10 carbon atoms or less;
in the general formula (3) and general formula (4)-2 above,
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less; and
in the general formula (4)-2 above,
X¹ is a halogen atom or a perfluoroalkanesulfonic ester group.

24. The method for producing an organic material according to claim 23, wherein the ring assembly constituting Ar² in the general formula (4)-2 is biphenyl, binaphthyl, or bianthracenyl.

25. A method for producing an organic material represented by a general formula (3) below, the method comprising reacting a compound represented by a general formula (5)-1 below with a compound represented by a general formula (5)-2 using a metal catalyst, wherein general formulas (3), (5)-1, and (5)-2 are as follows: 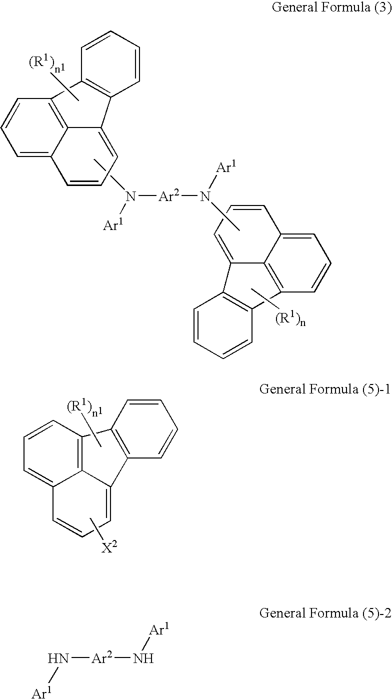
wherein:
in the general formula (3) and general formula (5)-1,
n¹ is an integer of 0 to 3, and
R¹ is an alkyl group having 10 carbon atoms or less;
in the general formula (5)-1,
X² is a halogen atom or a perfluoroalkanesulfonic ester group; and
in the general formula (3) and general formula (5)-2,
Ar¹ is a monovalent group which is derived from monocyclic or fused-ring aromatic hydrocarbon having 20 carbon atoms or less, and which optionally has a substituent having 10 carbon atoms or less, and
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less.

26. The method for producing an organic material according to claim 25, wherein the ring assembly constituting Ar² in the general formula (5)-2 is biphenyl, binaphthyl, or bianthracenyl.

27. A method for producing an organic material represented by a general formula (3), the method comprising reacting a compound represented by a general formula (6)-1 below with a compound represented by a general formula (6)-2 using a metal catalyst, wherein the general formulas (3), (6)-1, and (6)-2 are as follows: 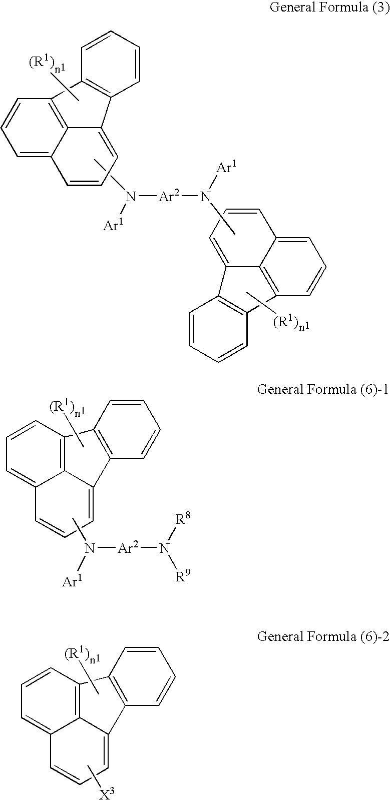
wherein:
in the general formula (3) and general formulae (6)-1 and (6)-2,
n¹ is an integer of 0 to 3, and
R¹ is an alkyl group having 10 carbon atoms or less;
in the general formula (3) and general formula (6)-1,
Ar¹ is a monovalent group which is derived from monocyclic or fused-ring aromatic hydrocarbon having 20 carbon atoms or less, and which optionally has a substituent having 10 carbon atoms or less, and
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less;
in the general formula (6)-1 above, R⁸ is a hydrogen atom or Ar¹, and R⁹ is a hydrogen atom; and
in the general formula (6)-2 above, X³ is a halogen atom or a perfluoroalkanesulfonic ester group.

28. The method for producing an organic material according to claim 27, wherein the ring assembly constituting Ar² in the general formula (6)-1 above is biphenyl, binaphthyl, or bianthracenyl.

29. A method for producing an organic material represented by a general formula (3), the method comprising reacting a compound represented by a general formula (7) below using an equivalent amount of a metal, a metal salt, or a metal catalyst, wherein the general formulas (3) and (7) are as follows: 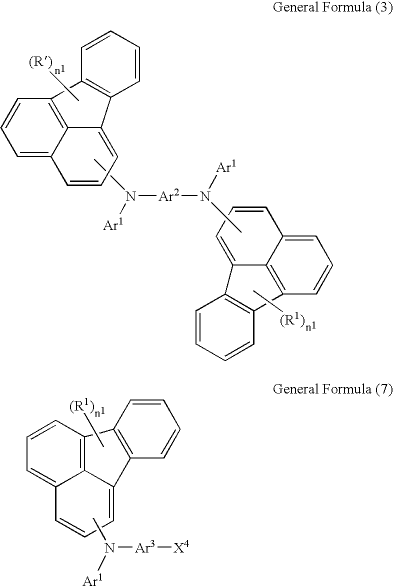
wherein:
in the general formula (3) and general formula (7),
n¹ is an integer of 0 to 3,
R¹ is an alkyl group having 10 carbon atoms or less, and
Ar¹ is a monovalent group which is derived from monocyclic or fused-ring aromatic hydrocarbon having 20 carbon atoms or less, and which optionally has a substituent having 10 carbon atoms or less;
in the general formula (3),
Ar² is a divalent group which is derived from a ring assembly having 30 carbon atoms or less and being comprised of monocyclic or fused-ring aromatic hydrocarbon having 1 to 3 rings, and which optionally has a substituent having 4 carbon atoms or less; and
in the general formula (7),
Ar³ is a divalent group which is derived from monocyclic or fused-ring aromatic hydrocarbon having 1 to 3 rings, and which optionally has a substituent having 4 carbon atoms or less, and
X⁴ is a halogen atom or a perfluoroalkanesulfonic ester group.

30. The method for producing an organic material according to claim 29, wherein the compound represented by the general formula (7) above is reacted with a compound corresponding to the compound represented by the general formula (7) wherein X⁴ is changed to magnesium halide, boric acid, or borate.

31. The method for producing an organic material according to claim 29, wherein, in the general formula (7), Ar³ is a divalent group derived from benzene, naphthalene, or anthracene.
